(19) United States
(12) Patent Application Publication
(10) Pub. No.: US 2005/0089458 A1
(43) Pub. Date: Apr. 28, 2005

(54) Refrigeration purifiers

(76) Inventor: Simon Forbes Oke, Wingfield (AU)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/494,290

(22) PCT Filed: Nov. 4, 2002
(86) PCT No.: PCT/AU02/01479

(30) Foreign Application Priority Data
Nov. 2, 2001 (AU) PR 8614

(51) Int. Cl. A61L 2/18; A61L 2/20

(52) U.S. Cl. 422/207; 422/1; 422/4; 422/5; 422/28; 422/123; 62/78

(57) ABSTRACT

A method and apparatus for the continuous or periodic cleaning and purification of water or air or surfaces in refrigeration systems, such as ice machines and refrigerated containers. Oxidants and oxidant radicals are produced electrically in a stream of air and the resultant gas is injected into a stream of water or air which flows through the refrigeration system and where further oxidants may be generated in this downstream flow of water or air.

Refrigeration Purifiers are products, which control the quality of air, water and surfaces in refrigeration systems. These systems comprise a refrigeration unit or plant, which is used to cool air or water or produce. The refrigeration unit may be an integral part of a refrigeration machine (such as an Ice Machine) or may be used to create a refrigerated space (such as a Cool Room),.

### BACKGROUND OF THE INVENTION

Refrigerated machines include Ice Machines, Drinking Water Coolers and Water Fountains. Such machines contain water and various electrical and mechanical components. The water may be recirculated and/or may interface with air. Therefore pollutants may build up in the liquid or frozen water, on component surfaces and in air spaces either within the machine or at exit points from the machine. Pollutants can include microorganisms, organic load, scale, off-odours, off-tastes and off-colours. Some refrigerated machines purge water to control pollutants and therefore water wastage occurs.

Refrigerated spaces include cold rooms, cool rooms, refrigerated ship containers, refrigerated trucks, commercial refrigerators and residential refrigerators. Such spaces contain air and produce. The air is generally recirculated around the space and repeatedly through the refrigeration unit. The air is cold and therefore water vapour tends to condense, creating a moist environment. Pollutants may build up in the air, on produce surfaces and in water aerosols. Pollutants can include micro-organisms, organic load, ethylene and scale. Some produce is typically wasted in refrigerated spaces.

Refrigerated systems, such as refrigerated machines and refrigerated spaces need to be kept clean. Cleaning is also referred to as purification or sanitation or disinfection. Cleaning may be periodic or continuous.

An example will now be given of an indicative refrigerated machine, namely an Ice Machine. Ice Machines are products which use refrigerants to cool water to create ice. They are used in houses, commercial premises and industrial premises. In some applications they are connected to ice dispensing machines or post-mix syrup machines or drink dispensers to supply the ice to various locations. They are common throughout the world in hotels, clubs, commercial kitchens, pubs, restaurants, bars, home refrigerators and industrial premises.

An Ice Machine is shown conceptionally in FIG. 1.

The Ice Machine comprises three main chambers. The first chamber may be called the refrigeration unit 2 and comprises the components which generate cold conditions such as a compressor, refrigerant, coolant coils and so on. The second chamber is adjacent to the first and may be called the ice rack chamber 3. This chamber comprises the ice racks 4, a fluid transfer device 5 (in this case a water pump), a water reservoir 6, and a pipe 7 which has water delivery holes 8. Water regularly circulates within the Ice Machine. The third chamber is the hopper 9, and may be located beneath the first two chambers. The ice machine is of course connected to a water supply 10, which may be filtered 11, either close to the machine, or where te mains water supply enters the premises. Some water may exit the system as “bleed or dump or purge water” so as to remove pollutants through an exit pipe 1. Many variations of this layout are possible.

A typical mode of operation for an ice machine is now described. An ice making cycle may take 10 to 60 minutes for example and begins when water enters the machine, determined by the various controls, which may operate watervalves. This water fills up the reservoir 6. The water pump 5 then pumps water up the pipe 7 and the water runs out holes 8 and down, under gravity, over the ice racks 4. Therefore the water flows through air. The water is then captured in the reservoir again and recirculated. The components in the refrigeration chamber serve to cool the ice racks 4 and thus ice is produced. The ice may then be released into the hopper 9, either via a mechanical movement or by a reverse cycle refrigeration system, whereby the ice racks are temporarily heated. A sensor in the hopper may control the operation, based on the ice supply and demand rates. Many variations of this operation are possible.

An example is now given of an indicative refrigerated space, namely a refrigerated container. Refrigerated containers are used to transport food produce on ships, trains, aircraft and trucks. They comprise a refrigeration unit attached to one end of a container. Air recirculates throughout the space and repeatedly through the unit, where it is cooled.

A refrigerated container is shown conceptionally in FIG. 2.

The refrigerated container contains two main chambers. The first chamber may be called the refrigeration chamber 2 and comprises the components which generate cold conditions such as compressor, refrigerant, cooling coils and so on. The second chamber is adjacent to the first and may be called the Produce Chamber 3. This chamber comprises produce, insulation, an access door and so on. A fluid transfer device 5, (in this case an air fan) causes air to recirculate between the two chambers. Some air may exit the system as waste air so as to remove pollutants, including ethylene through an exit vent 1. Many variations of this layout are possible.

A typical mode of operation for a refrigerated container is now described. Air is sucked from the top of the produce chamber 20, through the fan 5, and into the refrigeration chamber 2 where it is cooled and possibly dried as it passes downwards through the cooling coils. It then is forced from the base of the refrigeration chamber 21 back into the produce chamber where it flows through flutes and then upward 22 to cool the produce. Many variations of this operation are possible.

The art concerns air, water and surface quality control devices in refrigeration systems where water or air are always present as working fluids and are cooled. Therefore the quality of the water and the air are important for reasons of human health and safety and they are also important for reasons regarding effective operation of the refrigeration system.

The various pollutants and cleaning responses, which are common to various refrigeration systems, are now described:

Various pollutants enter the refrigeration machine or space either through water or air or human contact or the presence of produce. If the supply of air or water is not correctly filtered, then air or water or surface pollutants may exist. Consequently, pollution can build up inside the refrigeration machine or space. This includes bio film or bio slime. Other microbes can also build up such as bacteria, viruses, algae, fungi and protozoa. Other pollutants include salts and scale, odour, off-colours and off tastes. These pollutants can cause the water or produce itself to be unhealthy or unpalatable or unhygienic. The bio film is unsightly and in some instances can cause visible black flakes to be deposited in ice or water.

In the case of refrigerated spaces, which contain food produce, it is important to maximise shelf life and to minimise food spoilage. Spoilage is caused by surface microbes on the food produce and/or by the generation of ethylene from the food produce as it ripens, which in turn, causes faster decomposition. Such refrigerated spaces may exit some air from the space to remove ethylene, but this reduces cooling efficiencies. Other refrigerated spaces are filled continuously with chemicals or modified atmospheres in order to increase shelf life, but this is expensive.

On some premises, yeast is present. Examples are premises where bread is made in the kitchen. In such cases, the degree of bio film can be excessive. Large films can occur, caused by airborne yeast entering the refrigeration machine. This is especially a problem with ice machines.

Most organisations clean refrigeration machines and spaces between once per week and once per year. The word “clean” may be used to refer to the removal of deposits and debris and scale from the inside of the machine or space. The word “purify” may be used differently to refer to the killing of microbes on the inside of the machine or space. Therefore it is necessary to achieve both objectives—to clean and to purify.

Refrigeration cleaning fluids and processes are well known. Most use liquid chemicals or aerosols.

A common process is as follows. The organisation purchases chemicals, which can include caustic based solutions, detergents, defoamers, chlorine compounds, chelating agents, alkali salts, iodine, etc. The refrigeration system is partly disassembled and these cleaning fluids are manually applied and scrubbing takes place. This is followed by rinsing to remove chemical residue, which would otherwise affect the taste of the water or ice or produce. Contract labour may be used for this process. Hazardous chemicals may be used.

Various products and special equipment are also available, which include automatic cleaning. Such devices attach to ice machines, for example, as accessories. Some use liquid chemicals, which are fed into a chemical dosing device such as a centrifugal pump. The devices often include valves and timers or other control devices. Some use chemical devices, which release gases or aerosols into the air space. The chemicals can include those listed in the previous point.

The chemicals are consumables and therefore they need to be frequently purchased, transported and stored and dispensed. This creates on-going purchasing and logistics costs. The chemicals may be hazardous in nature.

This creates occupational health and safety problems, during transport, storage and handling. Handling may include the need to pour between vessels and to dilute with water. The chemicals may require a rinsing stage after the refrigeration system is cleaned. If rinsing is incomplete, the ice may suffer “off-tastes” or may be unhealthy. The chemicals and pollutants, following cleaning may need to be removed from the refrigeration system and disposed of. Typically they should not be run to the drain or sewer, because they may be toxic. If they are toxic, then alternative disposal costs are high. If they are run to the sewer, then the operator is liable to be breaking the law. The chemicals may require a soaking time. Therefore labour costs of the operator can be high. Some chemicals do not clean efficiently or purify efficiently. Some chemicals are excessively corrosive, and this is amplified where soaking times are required.

If salts exist in the feed water for a refrigeration machine then the concentrations of those salts may build up over time in the ice machine. The salts may deposit on surfaces (both wetted surfaces and dry surfaces), thus forming scale. This scale can cause moving parts to foul and can cause corrosion. This reduces the life of components, increases the need for servicing, increases failure rates, and causes aesthetic problems such as the formation of white stains, etc. It can also reduce the cooling efficiency of the refrigeration components and increase electricity usage. The response is to continuously or periodically dump or purge water from an ice machine, for example. In this way, unacceptable concentrations of salt or other pollutants may be exited from the system. However, dump or purge water is not a complete solution, and it leads to expensive water usage and water wastage.

### OBJECTS OF THE INVENTION

It is an object of this invention to overcome one or more of the above problems associated with the control of air, water and surface quality in refrigeration systems and the cleaning and purifying of such systems.

A further object of the invention is to provide a system for controlling the quality of air and water and of wetted and dry surfaces in refrigeration systems in which consumables are not required to be purchased and in which no polluting materials are used.

### BRIEF STATEMENT OF THE INVENTION

Thus there is provided according to the invention a method of cleaning and purifying water and air and surfaces in refrigeration systems including the steps of electrically producing oxidants by passing air through an oxidising chamber such as a corona discharge chamber, mixing the oxidants with a flow of water and air whereby the oxidants cause contaminants in the system, including scale and micro-organisms and ethylene to be removed, oxidised, killed or flocculated and filtered.

Also there is provided according to the invention a method of cleaning and purifying water or air or surfaces in refrigeration systems, including the steps of producing ozone and/or hydroxyl radicals in the water or air, which flow through the refrigeration system to react with and remove contaminants in the water or air or surfaces.

Additionally there is provided, according to the invention, a method of cleaning and purifying water or air or surfaces in refrigeration systems including the steps of passing air which contains oxygen and water vapour through an oxidising chamber to produce one or more oxidants in the form of ozone, hydrogen peroxide, hydroxyl radicals, hydroxyl ions, atomic oxygen, and atomic oxygen ions and injecting and mixing the oxidants in the flow of water or air through the refrigeration system.

There is also provided according to the invention a method of cleaning and purifying refrigeration machines including the steps of partially dissolving oxidants in water and enabling some oxidants to vent from the water into the air, so that these oxidants react with and remove contaminants in the air space inside the refrigeration machine and on non-wetted surfaces of the refrigeration machine.

There is also provided apparatus for cleaning and purifying refrigeration systems, said apparatus including means of micro-flocculating salts in the water, producing a motive force by bubbling air through a friction tube in water, and passing this flocculated material and water through a water filter, thus capturing the salts.

### BRIEF DESCRIPTION OF THE DRAWINGS

In order to more fully describe the invention reference is now made to the accompanying drawings in which:

FIG. 1 is a view of a prior art Ice Machine.

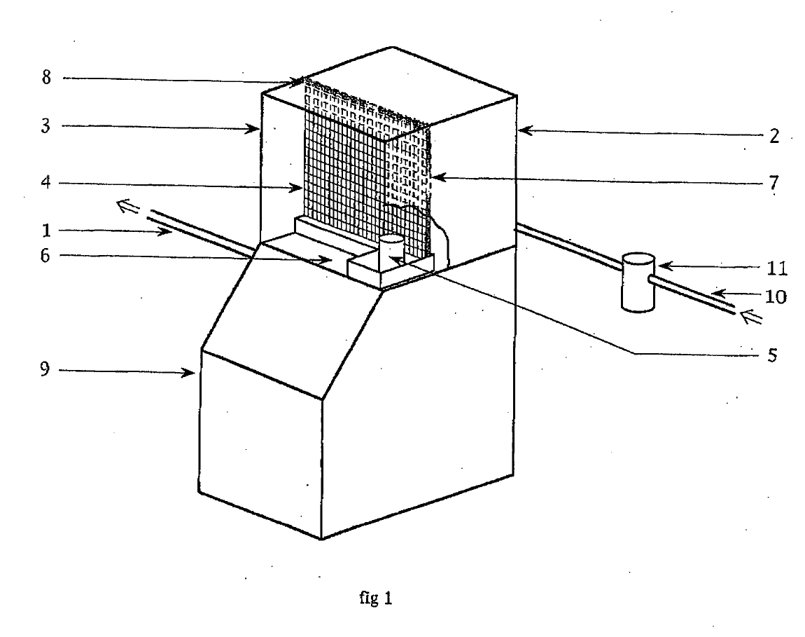

FIG. 2 is a view of a prior art Refrigerated Container.

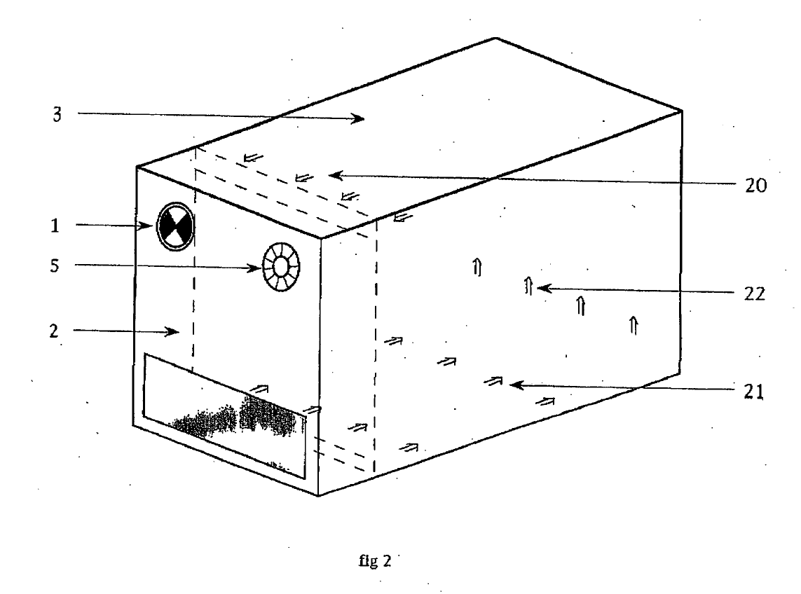

FIG. 3 is an example of a Refrigeration Purifier located in an Ice Machine.

FIG. 4 is an example of a Refrigeration Purifier located in a Refrigerated Container.

FIG. 5 is a compact form of the invention, including a diffuser.

FIG. 6 is a compact form of the invention, including a venturi.

FIG. 7 is an alternate form of the invention, including an oxygenator.

FIG. 8 is an alternate form of the invention, including a humidifier.

FIG. 9 is an alternate form of the invention, including a degasser.

FIG. 10 is an alternate form of the invention, including a friction tube and water filter.

### DESCRIPTION OF THE PREFERRED EMBODIMENT

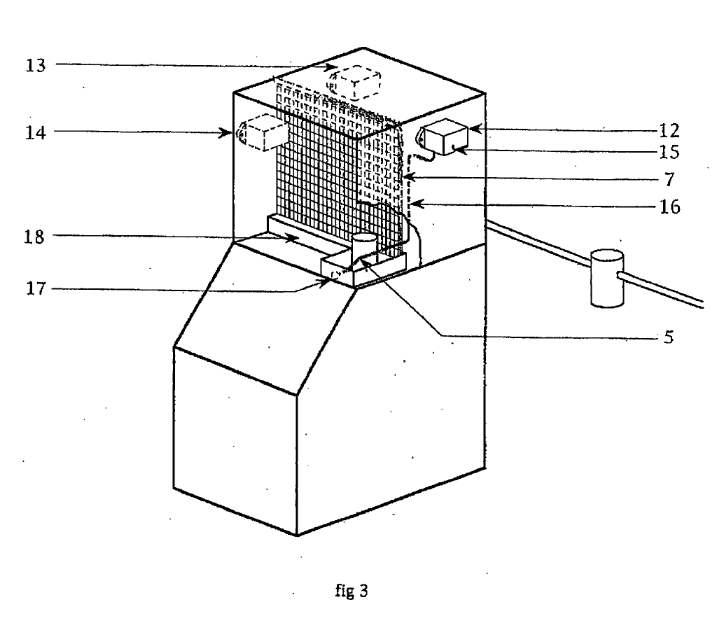

FIG. 3 shows an example of how the Purifier 12 can be located in a refrigeration machine, such as an ice machine. The Purifier can be fastened or hung in any position. It can be located on the outside of the chamber in position 12, to allow easy installation or it can be located inside the refrigeration chamber in position 13 or inside the ice rack chamber in position 14.

The Purifier has an inlet hole 15, which delivers air to it, preferably from a location where the air is relatively dry such as the outside of the product. A small tube and air filter may be attached to this inlet hole. The Purifier also has an outlet tube 16 to deliver the oxidants to the water, preferably in the reservoir 6, where it is dissolved in the water preferably by using a porous diffuser 17. Alternatively the diffuser can be a long device and located at position 18 in the reservoir under the ice tray. The Purifier is connected to an electrical supply, such as the terminals of the ice machine's water pump, or to a circuit which allows the Purifier to operate intermittently, or only when ice is being made.

Typically, the Purifier is on only when the ice machine is on arm making ice. In addition, the Purifier may be connected to any ice machine component, which cycles. For example, the water pump may turn on when mains water pressure has caused water to fill the reservoir. If the Purifier is electrically connected to the water pump, then it turns on only when the reservoir is full of water. The Purifier may have a timer, which causes it to operate for 5 minutes for example. The ice making cycle may be 20 minutes. Therefore the Purifier may only operate when the water level is highest, improving the dissolving of the oxidants and water. Such intermittent use will also increase the fife of the Purifier.

Not all of the oxidants will dissolve in the water for two reasons. First, when the diffuser is below the water level, some oxidant will dissolve but some will remain as bubbles and vent from the water surface to the air. Second, with some ice machines, the water level will vary, and the diffuser may protrude above the water level at times, in which case oxidants enter the air directly. In both instances, this is advantageous, because it is desirable to treat both the water and the air. By achieving or designing the Purifier so that some oxidants pass into the air, it is ensured that the gas phase oxidants reach all inner surfaces of the refrigeration chamber, both wetted surfaces and dry surfaces. The gas phase oxidants can also reach the inner surfaces of the hopper, and can pass between voids amongst the ice cubes in the hopper. Therefore the oxidants act to kill or control microbes on wet and dry surfaces of the ice machine chambers.

It is preferable that oxidants treat both the water, (so that the water is purified) and that they treat the air (so that non-wetted surfaces are also purified). This is achieved by placing the diffuser in the water, as it is then inevitable that some of the oxidants will vent from the water into the air and then be dispersed to surfaces. Alternatively, the diffuser can be placed in air, rather than in water, so that oxidants then flow through the air, to surfaces and some oxidants scrub into the water. The invention includes this alternative of placing the diffuser in the air rather than in the water.

Alternatively a venturi may be located in pipe 7 or a bypass connected to pipe 7, in which case the motive force from pump 5 (or from a new pump) causes the oxidants to be sucked through tube 16 and into the venturi and thus into the flow of water.

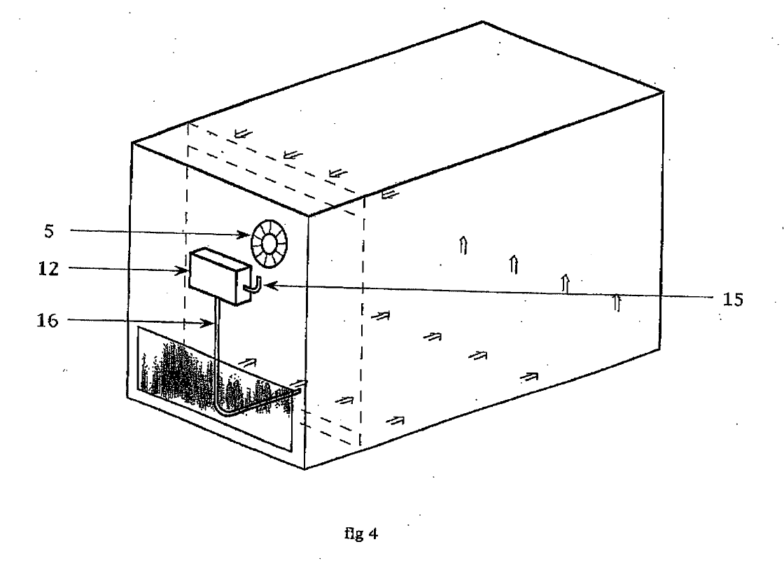

FIG. 4 shows an example of how the Purifier 12 can be located to serve a refrigerated space, such as a refrigerated container. It can be located on the outside surface of the refrigeration chamber, for easy access, but it can alternatively be inserted into this space so it is flush with the external surface. The Purifier has an inlet hole 15 to deliver air to it. The oxidant leaves through tube 16. The oxidants are discharged away from the Purifier and towards the downstream side of the airflow, so that there is no short-circuiting or return of oxidants to the Purifier inlet, within the refrigeration chamber itself. Preferably, the oxidants are delivered downstream of the cooling coils to minimise corrosion.

The Purifier 12 may contain an in-built air compressor to provide a flow of gas into inlet 15, through the Purifier itself and out of the tube 16. Or the Purifier may contain a further tube which connects inlet 15 to the vicinity of the fan outlet 5. By positioning this further tube close to the fan outlet, with variously shaped inlet cones and venturis, the fan can supply sufficient pressure to deliver the required small air flow rate through the Purifier. In the case of a low pressure axial fan, large tube diameters are necessary. Therefore it is possible for the Purifier 12 to not require an air compressor, as it instead utilises the airflow caused by the Refrigeration Systems own fan.

The Refrigeration Purifiers create strong oxidants from air. These oxidants are created by using electrical energy, such as by passing air, which may contain water vapour through a corona discharge field. The oxidants, which are created, may include one or more of the following: ozone (triatomic oxygen), hydroxyl radical, hydroxyl ion, hydrogen peroxide, atomic oxygen, atomic oxygen ion, diatomic oxygen ion, hydrogen ions, nitrogen ions and similar. These oxidants are then dissolved into water or mixed with air by using a contact mechanism such as a porous diffuser or a venturi. This mixture, of oxidants then flows through the refrigeration system. There may be a further phenomenon where ozone reacts with intermediary oxidants such as hydrogen peroxide and this creates further hydroxyl radicals downstream in the system.

The oxidised water may include:

i. Some oxidants, which are properly dissolved in water and are effectively in the liquid phase.

Some oxidants, which are resident in water but are not dissolved and are still in the gas phase, and for example may be seen as bubbles in water which vent from the water surface. Alternatively the bubbles can be removed and the captured gas then reinjected into the water before exiting the product.

Some oxidants, which are mixed, directly into air (such as with refrigerated spaces).

Some residual air (diatomic oxygen molecules and nitrogen molecules and water vapour) which are present because the efficiencies of the Purifier are less than 100%.

The oxidised water and air both cleans and purifies pollutants from the refrigeration system:

i. Cleaning is primarily by a process of oxidation of inorganic and none living organic substances in the water or air or on surfaces, and also by a process of killing micro-organisms which act as a substrate for other pollutants on the surfaces, and also by a process of friction where the oxidised water or air flows past the surfaces.

ii. Cleaning is also by a process of micro-flocculating salts in water and then capturing the flocculant in a water filter.

iii. Purification is by a process of the oxidants causing denaturing of the protein structure in micro-organisms and thereby killing them.

Typically the Purifier is operating whenever the refrigeration system is operating. Therefore the water and air treatment is continuous or semi-continuous.

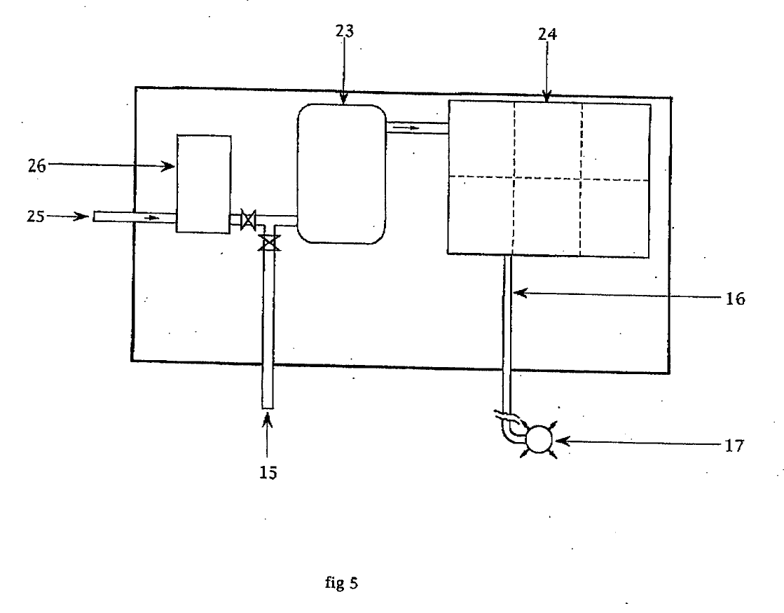

FIG. 5 shows a first embodiment of the invention, which is a compact Refrigeration Purifier. The air feedstock may be supplied from the ambient air through air inlet 15 and flow directly to a compressor 23 and then to an oxidising chamber 24. The ambient air contains some natural water vapour. Alternatively the air feedstock may be supplied from ambient air through alternative air inlet 25 and then pass through a dryer 26 so as to partially or completely remove the water vapour before it passes to the compressor and oxidising chamber. The oxidising chamber utilises a corona discharge field to create strong oxidants. The oxidants are in the gas phase and may also be in the aqueous phase as a vapour or liquid aerosol. They then pass through a tube 16 to a contactor 17. The contactor is preferably a porous diffuser which is remotely positioned in the reservoir of a refrigeration machine with water, or which is directly positioned in the recirculating air stream of a refrigerated space.

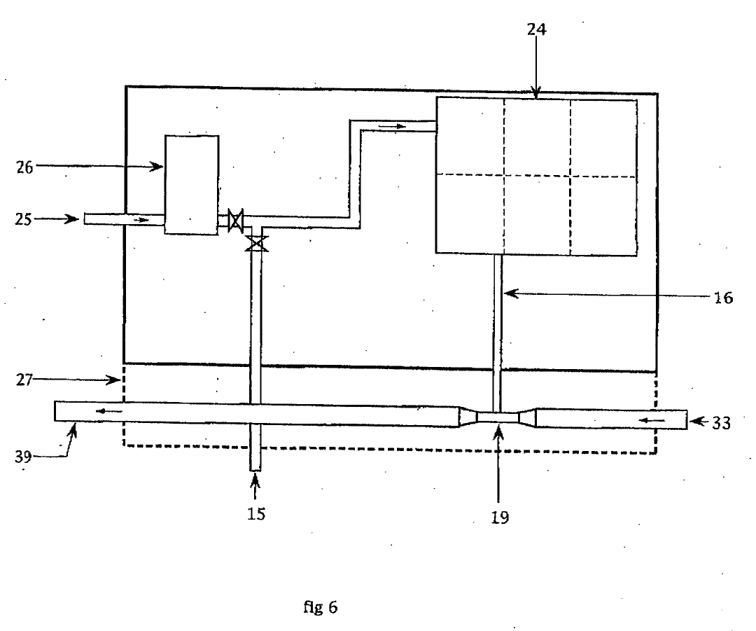

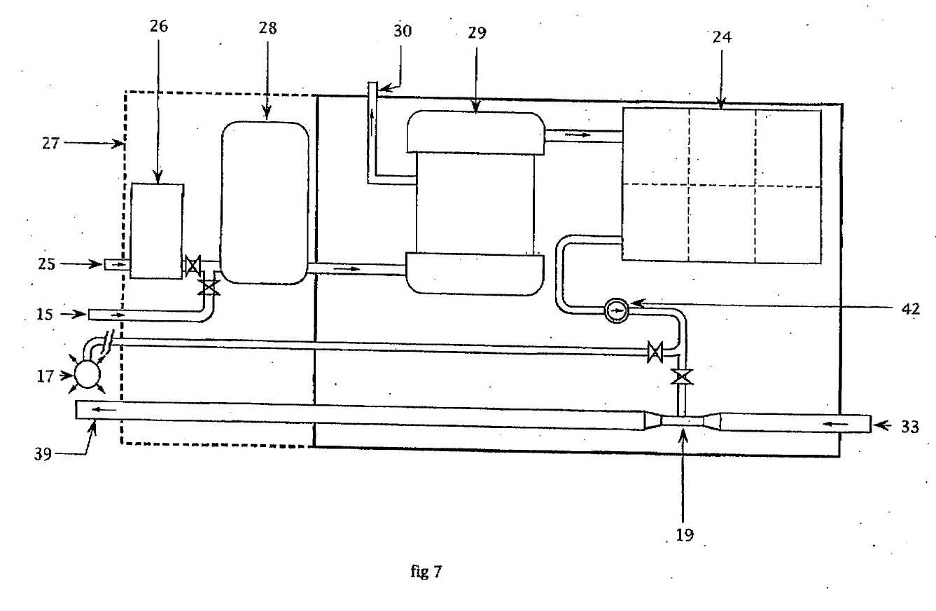

FIG. 6 shows a further form of the invention, which is also a compact Refrigeration Purifier. The apparatus is similar to FIG. 5 but the contactor is a venturi injector 19. The refrigeration systems' existing fluid transfer device (pump or fan) can then be used to provide the motive force. As the water or air flows through the venturi, a vacuum is created in the gas port of the venturi, which sucks gas through the oxidising chamber 24. Thus the compressor component is not required. In one version of the invention, the parts shown in FIGS. 5 and 6 are fully encapsulated in a potted mass. The dashed line 27 illustrates that the contactor device can either be included in this potted mass or can be located remotely. The dashed line has a similar meaning in FIGS. 7 to 9.

FIG. 7 shows a further form of the invention to increase the concentration of oxidants, by using an oxygen concentrator device, also known as an oxygenator. The product, is a larger unit to suit larger commercial and industrial refrigeration systems. The air feedstock may be supplied from the ambient air through inlet 15 and then passes through an air preparation system, such as a compressor 28 through a tube to an oxygenator 29 to remove nitrogen 30 and achieve a high oxygen concentration. Alternatively the air feedstock may be supplied from ambient air through alternative air inlet 25 and then pass through a dryer 26 before it passes to the compressor 28. The output from the oxygenator passes to the oxidising chamber 24. The remainder of the Purifier system is then as was previously described in reference to FIGS. 5 and 6. The contactor device can be a porous diffuser 17 or alternately be a venturi 19 or simply a hose tube outlet.

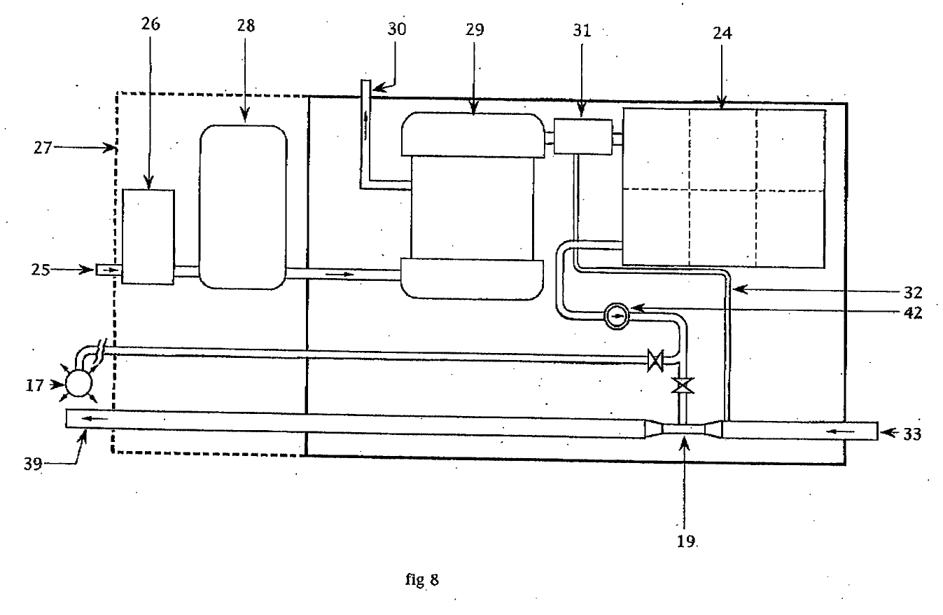

FIG. 8 shows a further form of the invention to create high concentrations of oxidants in the oxidant outlet, and also to optimise the efficiency and life of the product. The feedstock air enters the air inlet 25 and is dried 26 before being compressed 28 and oxygenated 29. The gas therefore mainly comprises dry oxygen. However before this gas flows into the oxidation chamber 24 it is humidified by a humidifier device 31. A flow of water is supplied from a separate source or bled from a main water flow through line 32 to the humidifier device 31 which mixes water in aerosol or droplet or vapour form into the gas which is flowing from the oxygenator to the oxidation chamber. The humidifier device preferably comprises a membrane contact device, which allows pressurised water to pass through small pores of a membrane and thus enter a flow of oxygen. Thus water vapour or aerosol (H2O) and oxygen (O2) and a minor quantity of residual air pass into the oxidation chamber. The remainder of the Purifier system is then as was previously described in reference to FIGS. 5 and 6. The contactor device can be a porous diffuser 17 or alternately be a venturi 19, or simply a hose tube outlet.

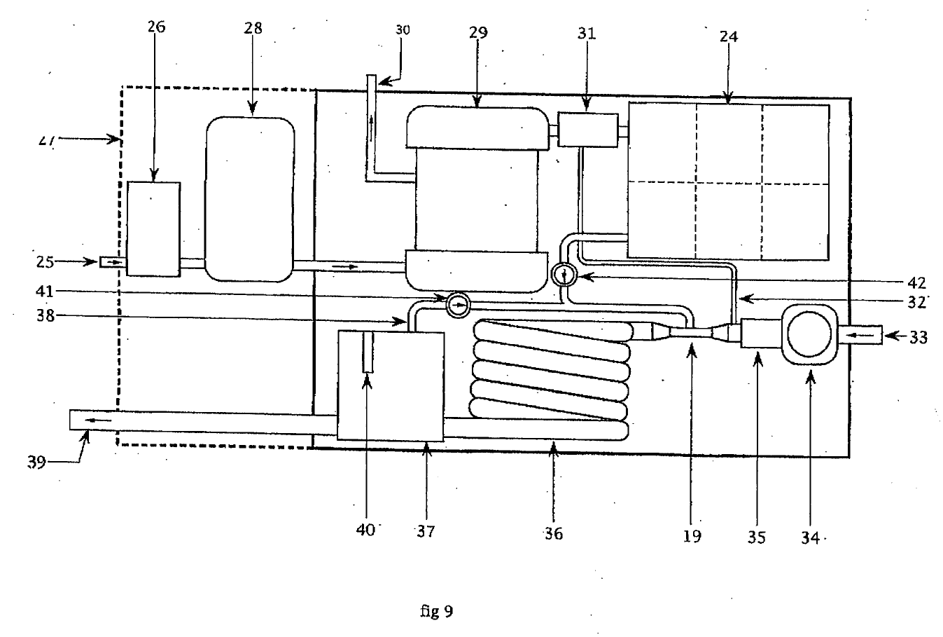

FIG. 9 shows a further form of the invention which includes efficient mixing, degassing and re-injection and applies to refrigeration machines which utilise water. The main water flow enters through water inlet 33 and then passes through water solenoid valve 34. The water feedstock may be from a pressurised mains system or it may be from a tank or dam in which case a water pump may be included with the Purifier product. The optional solenoid 34 can serve both as a backflow prevention device or as an automatic method of activating the Purifier's electrics when connected to a flow switch 35, from which it receives an electrical signal. After the oxidants have been contacted with the water, they are mixed in the water by the mixing coil 36.

The oxidised water in the mixing coil contains some oxidants which are dissolved and some which are still in the form of bubbles. This “undissolved” component would normally be wasted and would vent to air at the first opportunity. In the invention the oxidised water then passes through a degasser chamber 37 where the bubbles are separated from the water. The bubbles are expelled as gas to the vent outlet 38, whilst the oxidised water leaves the product at the oxidised water outlet 39. One form of the degasser chamber comprises a pressure vessel or tank into which the oxidised water enters, and thus the water velocity slows and allows bubbles to rise to the surface of the water, which creates a gaseous space at the top of the chamber. As this gas builds up, the water level in the chamber reduces and a float switch 40 sends an electric signal to a degasser solenoid 41 which opens and allows the gas to vent through tube 38, until the float switch moves the solenoid back to the closed position. The vent tube 38 contains ozone gas. Preferably it is connected into the gas line just downstream of check valve or solenoid 42, or connected to a second gas port on contactor 19, or into the gas port of a second contactor which may be positioned upstream or downstream of the first contactor. In this way the ozone gas is used efficiently.

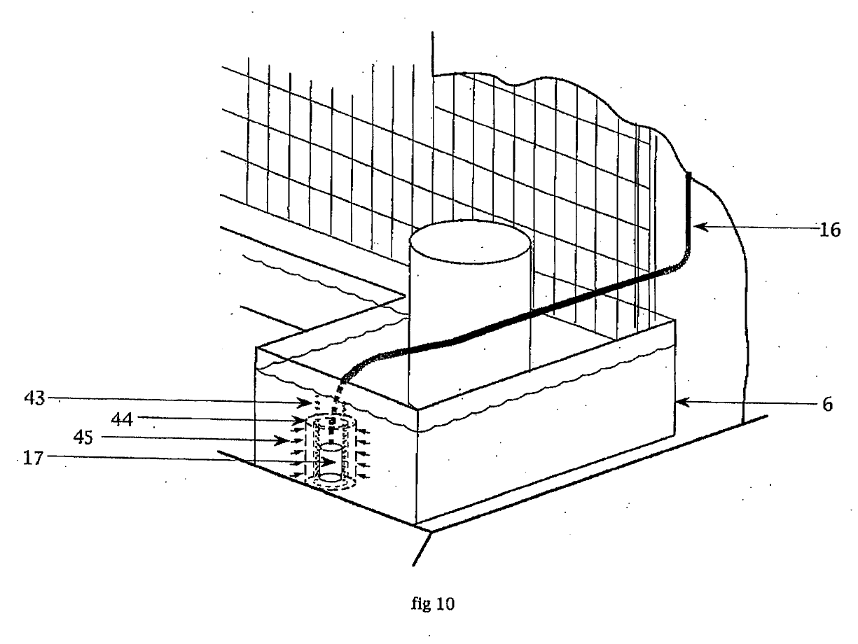

FIG. 10 shows an alternate variation of the invention where a special water filter and friction tube component is added to the refrigeration machine which utilise water. This component removes certain pollutants or substances from the water. In particular it can remove “flocculated salts” from the water. The oxidants cause the salts to undergo a process of micros flocculation as follows to change the salt component from a dissolved form to an un-dissolved form. The oxidants in the water cause organic pollutants in the water to become polar. These polar pollutants then bond with salts to create complex organic/inorganic compounds. These compounds flocculate from the water and can be captured in a filter. This process may be enhanced by using a cyclical timer to deliberately create partial oxidation by repeatedly turning the Purifier on and off, so that the oxidant concentrations in the water vary with time and it is ensured that for at least some of this time, the levels are such that partial oxidation occurs, as distinct from complete oxidation.

Preferably, a water filter of suitable micron size is placed in the water to filter the micro-flocculants out of the water. All that is then further required is a motive force to cause the water to pass through the filter. This can be a stand-alone pump or it can be the existing main water pump in the refrigeration system. Or the innovation described in the following point can be utilised.

A friction tube may be used to cause the water to flow, through this filter in a cost-effective manner, which does not require a further component to provide this motive force, as now described. The Purifier typically already includes an air compressor or air pump which delivers the oxidants through a tube 16 to a porous diffuser 17 located in the water reservoir 6. This porous diffuser emits gas bubbles 43 from its pores, some of which dissolve into the water and some of which rise quickly to the water surface. The diffuser 17 is located inside a cylindrical cartridge filter 44. In one variation of the invention, a friction tube or tubular shape may be further located on the inside of this cartridge filter. As the gas bubbles rise upwards out of the cartridge, a friction effect takes place which causes water 45 to be entrained into the filter. Thus water is sucked from the outside of the cartridge to the inside, at a low flow rate but on a continuous basis. Therefore there is an effective “water pump and water filter” in the refrigeration system, which is cost effective because the motive force for the water movement is created by using a flow of pressurized gas already existing in the Refrigeration Purifier. By changing or cleaning the water filter on a periodic basis, such as when the refrigeration system receives a general service, it can be seen that the salts have been flocculated, filtered and then removed from the system entirely.

It has been proved by tests and investigation that the invention as described above can create two sets of oxidants depending upon the air inlets used.

i. In FIGS. 5 and 6, when air inlet 25 is used, the feedstock air is dried, water vapour is removed and oxygen and nitrogen remain. Thus the resultant feedstock does not contain hydrogen atoms. The main oxidant then created by the oxidising chamber is ozone in medium concentrations.

ii. In FIGS. 5 and 6, when air inlet 15 is used, the feedstock air contains water vapour, oxygen and nitrogen. The main oxidants created by the oxidising chamber are ozone in the gas phase in medium concentrations, and also hydrogen peroxide in the aqueous phase and hydroxyl radicals, both in significant concentrations.

iii. In FIG. 7, when air inlet 25 is used water vapour is removed by the dryer, nitrogen is removed by the oxygenator, and high concentrations of oxygen remain. The main oxidant then created by the oxidising chamber is ozone in high concentrations.

iv. In FIG. 7, when air inlet 15 is used, nitrogen is removed but water vapour and high concentrations of oxygen remain. The main oxidants created by the oxidising chamber are ozone in the gas phase in high concentrations, and hydrogen peroxide in the aqueous phase and hydroxyl radicals, both in medium concentrations.

v. In FIG. 8, air inlet 25 is used. The dryer removes water vapour, the oxygenator removes nitrogen and high concentrations of oxygen remain downstream of the oxygenator 29. The humidifier 31 then adds water vapour in a fine aerosol form. The main oxidants created by the oxidising chamber are ozone in the gas phase in high concentrations, and hydrogen peroxide in the aqueous phase and hydroxyls, both in high concentrations.

The advantage of creating hydroxyl radicals is that they are very strong oxidants and provide an advanced oxidation process. For example, measured in volts, the oxidation potential of chlorine gas is 1.36, ozone is 2.07 and the hydroxyl radical is 2.80. There are many substances, including some synthetic and natural organic chemicals, which have a slow reaction rate with ozone but a fast reaction rate with hydroxyls, and in such instances hydroxyls are superior oxidants. Hydroxyls have a short half-life, being a fraction of a second whilst ozone has a longer half-life, being up to 30 minutes in clean water. Therefore for micro-organism disinfection, where a residual oxidant level is required for a period of time, ozone is a superior oxidant. Other examples also exist where either hydroxyls or ozone or both, can be chosen to provide the optimum oxidant regime.

Further, the invention is able to create hydroxyl radicals in the downstream flow of water or air. In FIG. 8 for example, wet oxygen is used as feedstock to the oxidising chamber which creates ozone in the gas phase and hydrogen peroxide in the aqueous phase, and also creates some hydroxyl radicals. The ozone and the hydrogen peroxide are created independently from each other and at the same time and in a single operation, whilst the feedstock is passing through the discharge gap in the emitter. The ozone and hydrogen peroxide are then mixed into the main water or airflow at the contactor. The hydrogen peroxide then acts as an intermediary. It gradually reacts with some of the ozone, in this downstream flow, to create further hydroxyl radicals. Thus the invention provides ozone and hydroxyl radicals which are created or generated in the downstream flow, such as in the reservoir or in the distribution pipe work or the recirculating air refrigeration system. If the hydroxyls were only created in the oxidising chamber itself, then they would not be able to do useful work in downstream water or airflow, as they would disappear quickly due to their short half-life which is a fraction of a second. But because the invention allows them to be generated in a downstream flow, this limitation is solved, and the oxidants can act upon a larger downstream body of water or air and also upon surfaces of the refrigeration equipment.

Ozone decomposes in water or air with a natural half-life. When it does so, hydroxyl radicals are generated as a transient by-product. However the process described above, involving hydrogen peroxide, is a separate phenomenon and involves the generation of larger quantities of hydroxyl radicals from a reaction between ozone and hydrogen peroxide.

The presence of water vapour in the discharge space of the oxidising chamber acts to reduce the ozone output rate and the ozone concentration which would otherwise be achieved if the space was dry. However this effect is counteracted by the formation of hydrogen peroxide which in turn enables the generation of larger quantities of hydroxyl radicals.

The oxidising chamber is designed so that it can create ozone and hydrogen peroxide and hydroxyls, by receiving wet (humid) air or wet (humid) oxygen. A corona discharge field is developed. Mains electrical input is transformed into the optimum combination of voltage, frequency and wave shape, so as to disassociate the diatomic oxygen and water vapour molecules into atomic oxygen and hydrogen, to then enable recombination into the required oxidants.

The invention is designed to minimise corrosion rates and to extend component life, for applications where hydroxyl radicals and/or ozone are required and therefore wet air or wet oxygen feedstock is used:

i. In FIGS. 5 and 6, when inlet 15 is used, water vapour and nitrogen flow through the corona field in the oxidising chamber. Trace levels of substances may form, such as nitric acid, which may gradually corrode the surfaces of components in the oxidising chamber which are in the gas stream, including stainless steels. The oxidising chamber is designed so that it is non-corrosive. The oxidising chamber comprises emitters, power sources, printed circuit boards, etc. There may be multiple emitters, in parallel or in series, so as to achieve the desired oxidant output and concentrations. A corona field is created in the emitter and the feedstock flows through this field. The emitters include a high voltage electrode, an earthed electrode and a dielectric. The electrodes may be made of metals including stainless steels or other materials which are electrically conductive and such materials are corrosive to some extent. The dielectric is made of silicon or mica or epoxy filled glass or ceramic based materials, including glass, which have high corrosion resistance. The emitter design is laminated so that the dielectric lies on top of the high voltage electrode, or the high voltage electrode is encapsulated in a dielectric. Therefore this electrode is not adjacent to the feedstock flow and thus it does not corrode. In addition, or alternatively, the earthed electrode can also be laminated by positioning a second dielectric against it, or it can be encapsulated by the dielectric. Thus one or both electrodes can be completely removed from the feedstock flowing through the emitter and thus corrosion is reduced and the efficiency of the oxidising chamber is maintained.

ii. In FIG. 7 there is an oxygenator which removes nitrogen and thus substances such as nitric acid do not form in the oxidising chamber and thus corrosion is controlled. However, in the case of inlet 15, the water vapour flows through the oxygenator which can damage the molecular sieve media in it and reduce media life or reduce the efficiency of oxygen concentration. The oxygenator is designed so that it includes an excessive amount of molecular sieve media, and where this media may be easily replaced at a regular service interval.

iii. FIG. 8 shows a preferred configuration of the Refrigeration Purifier. The dryer 26 removes water vapour so that the molecular sieve material in the oxygenator 29 is not damaged and so that oxygen concentration efficiency is maintained. The oxygenator removes nitrogen so that substances such as nitric acid do not form in the oxidising chamber. The water vapour is added to the system at the optimum location, namely the humidifier 31, so that hydroxyl radicals can be created either in the oxidising chamber itself or in downstream flow via the hydrogen peroxide intermediary. The oxidising chamber can also utilise an emitter design with laminated electrodes as previously described, so as to provide an extra level of corrosion protection.

The invention may be configured by using various component options and configurations, including:

A timer device may be connected to cause the device to cycle on and off. In one variation of the invention, this cycling operation can make an important contribution towards achievement of the benefits obtained, by achieving the partial oxidation of organic pollutants which then combine with salts and result in the process known as micro-flocculation. The dryer component 26 may comprise desiccant media, with or without a regenerative heater circuit, or may be a refrigerative dryer, or may be a coelescer or water trap device or mist filter. A particulate filter may be added to remove pollutants to protect the compressor and oxygenator and oxidising chamber. The oxygenator may utilise a molecular sieve, or pressure swing absorption design, or membrane design. The compressor may be a rotary or reciprocating device or an air pump or a diaphragm pump. The compressor 28 and oxygenator 29 may be replaced with bottled oxygen. The humidifier may utilise a porous membrane or any other method which allows the oxygen to become partially or fully saturated with water. The oxidising chamber may comprise corona discharge, plasma discharge, silent electrical discharge, dielectric barrier AC discharge or ultra-violet radiation or other electrical methods of creating oxidants. The oxidising chamber may include a catalyst such as Titanium Dioxide, with or without electrical potential applied to the catalyst surfaces. The emitters in the oxidising chamber may comprise electrodes which are tubular in shape or which utilise a parallel plate shape. The electrodes may be solid material or may be granular. The contactor may comprise a venturi, or a porous diffuser which bubbles into a basin or contact tower or pipe, or a membrane device. Or the contactor may utilise a peristaltic pump through which the oxidised gas passes so that this pump forces the gas through a porous diffuser into the water flow. Or if mains water pressure is not used, then a dual head peristaltic pump may be utilised, where one pump head creates pressurised water for the purpose of the main water flow and the other head creates pressurised oxidised gas which is then forced through a porous diffuser into the water flow. The mixing coil may be replaced by or used in conjunction with a static mixing device placed in a section of pipe. The product can be configured with or without the alternative air inlets previously described, and preferably would only incorporate the inlets which result in hydroxyl radicals and ozone being created, including hydrogen peroxide as an intermediary, rather than ozone alone. The oxidising chamber may include multiple emitters and these emitters are preferably each encapsulated in a potting compound such as epoxy. This provides a method of achieving low electrical magnetic interference, safe electrical insulation and waterproofing. Cooling fins may be moulded into the cast potted shape.

The advantages of the invention include the following:

i. Hydroxyl radicals and ozone are created in the downstream water or airflow in the refrigeration system. Thus these oxidants can do useful work such as cleaning and purifying the main body of water and air and surfaces of the refrigeration system. The hydroxyl radicals are created in the refrigeration system itself, due to a reaction between the ozone and intermediary oxidants such as hydrogen peroxide, which are previously created in the oxidising chamber of the product and then mixed into the main water or airflow. The hydroxyl radicals are very strong oxidants which are ideal for oxidising inorganics and non-living organics whilst the ozone creates a temporary residual oxidation level which is ideal for killing micro-organisms.

ii. The process is an all-electric advanced oxidation process. There are no chemicals or consumables. This creates significant on-going purchasing and logistics savings. The combination of this all-electric process together with hydroxyls being generated in the downstream air or water (as per point i above), is a unique and innovative combination.

iii. Scale build up on surfaces (also called salts or slime or bio-film) is reduced. This includes mineral scale and organic film. This can reduce fouling of moving parts and can reduce corrosion. This increases the life of components, reduces the need for servicing, reduces failure rates, reduces aesthetic problems such as the formation of white stains, etc. It can also increase the cooling efficiency of the refrigeration components and reduce electrcity usage.

iv. Ethylene is oxidised by the mixed oxidants in the case of refrigerated spaces which contain food produce. This increases shelf life and reduces spoilage.

v. There are no hazardous chemicals. This creates occupational health and safety advantages, and logistics advantages during transport, storage and handling.

vi. The oxidants clean efficiently and purify efficiently. A wide range of micro-organisms are killed, including Pseudomonas and E Coli bacteria and Giardia and Cryptosporidium protozoa.

vii. Unpleasant odours are reduced.

viii. The colour and taste of water or ice improves. The water and ice are pure and hygienic.

ix. Water usage reduces which is “environmentally friendly”. This occurs because purge water can be reduced or eliminated entirely. Similar, purge air is reduced from refrigerated spaces, thereby increasing cooling efficiencies.

x. Running costs can be reduced clue to lower water and air usage, and reduced regular maintenance. This is due to purge water and air being reduced or eliminated entirely, and also due to less scale build-up and corrosion.

xi. The oxidants do not excessively corrode the refrigeration system fittings. Corrosion rates can be less than occur in the case of chlorinated mains water.

xii. Instruments can be used to give a sufficiently precise indication of whether the oxidation process is taking place.

Thus it can be seen that the quality of water, air and surfaces in refrigeration systems can be effectively controlled, and can be continuously cleaned and purified without the use of chemicals. By connecting a unit which provides an advanced oxidation process and passing the oxidised water or air through the refrigeration system, an effective and safe system of cleaning and purification is provided. In addition the invention can be applied to any equipment using recirculating liquid or gas in residential or commercial or industrial refrigeration processes.

Although alternate forms of the invention have been described in some detail it is to be realised the invention is not to be limited thereto but can include variations and modifications falling within the spirit and scope of the invention.

1. A method of cleaning and purifying water or air or equipment surfaces or food surfaces or foodstuffs in refrigeration systems, including the steps of producing oxidants in a fluid (including water and/or air) which is a part of the refrigeration system to react with and remove contaminants in the refrigeration system, where these oxidants are generated from molecules of air and/or water and thus contain oxygen and/or hydrogen atoms only, and include oxidants other than ozone, such as hydroxyl radicals or hydrogen peroxide.

2. A method of cleaning and purifying water or surfaces or air in refrigeration systems, including the steps of providing a flow of fluid as a part of the refrigeration system, passing air which contains oxygen and water vapour through an oxidising chamber to produce oxidants in the form of hydrogen peroxide and one or more of hydroxyl radicals, ozone, hydroxyl ions, atomic oxygen, and atomic oxygen ions and injecting and mixing the oxidants in a flow of fluid (including water or air) within the refrigeration system.

3. A method of cleaning and purifying water or surfaces or air in refrigeration systems wherein ozone and hydrogen peroxide are produced in an oxidising chamber and then injected into water or air wherein the hydrogen peroxide then acts as an intermediary and reacts with the ozone to form hydroxyl radicals downstream of the point of injection into the flow of water or air, including in the refrigeration system through which the oxidised water or air flows.

4. A method of cleaning and purifying water or surfaces or air in refrigeration systems as defined in any one of claims 1, 2 or 3 including the step of generating the oxidants by an electrical means only.

5. A method of cleaning and purifying water or surfaces or air in refrigeration systems as defined in any one of claims 1, 2 or 3, including the steps of passing air through an ozone generator, then injecting and mixing the ozone into the water or air flowing through the refrigeration system in order to clean and purify it.

6. A method of cleaning and purifying water or surfaces or air in refrigeration systems as defined in any one of claims 1, 2 or 3, including the steps of drying and compressing air, passing the dried compressed air through an oxygenator to remove nitrogen from the air, then adding water in the form of aerosol or vapour or mist or droplets into the gas, passing this gas which has high concentrations of oxygen and water vapour through an electrical oxidising chamber and injecting the resultant oxidants into the flow of water or air.

7. Apparatus for cleaning and purifying refrigeration systems as defined in any one of claims 1, 2 or 3, said apparatus including an air inlet, an oxidant or ozone generator having an inlet connected to the air inlet, and an outlet connected to a passage between the water or air inlet and outlet whereby the products from the oxidant or ozone generator are passed into and mixed with the water or air to clean and purify a refrigeration system.

8. Apparatus as defined in claim 7 characterised by an oxygenator positioned in the air line prior to the oxidant or ozone generator whereby oxygen enriched air is passed to the oxidant or ozone generator to produce ozone and/or hydroxyl radicals generated down stream in the water or air flow.

9. An apparatus as defined in claim 7 or claim 8 characterised by an air drier positioned in the air inlet line.

10. Apparatus for cleaning and purifying refrigeration systems as defined in any of claims 1 to 9, said apparatus characterised by an inlet air tube which is connected to, or in the vicinity of, the outlet of an existing recirculation fan, so as to pressurise the inlet air of the apparatus and cause airflow through the oxidation chamber of the apparatus.

11. An apparatus as defined in 8 characterised by a humidifier positioned in the gas line between the oxygenator and the oxidant or ozone generator to humidify the gas by water spray, water aerosol, mist, droplet or steam.

12. Apparatus for cleaning and purifying refrigeration systems as defined in any of claims 1 to 11, said apparatus characterised in that the oxidised water or air flow passes through a mixer prior to entering the refrigeration system.

13. Apparatus for cleaning and purifying refrigeration systems as defined in any of claims 1 to 12, said apparatus characterised by passing oxidised water flow through a degasser to remove undissolved gases and to reinject those gases into water flow prior to exiting the apparatus and entering the refrigeration system.

14. An apparatus for cleaning and purifying refrigeration machines as defined in any of claims 1 to 13, including the steps of partially dissolving oxidants in water and enabling some oxidants to vent from the water into the air, so that these oxidants react with and remove contaminants in the air space inside the refrigeration machine and on non-wetted surfaces of the refrigeration machine.

15. Apparatus for cleaning and purifying refrigeration systems as defined in any of claims 1 to 14, said apparatus including means of micro-flocculating salts in water, producing a motive force in water by bubbling air through a friction tube in the water, and passing this flocculated material and water through a water filter, thus reducing the concentration of the salts in water.

16. Apparatus for cleaning and purifying refrigeration systems, as defined in any of claims 1 to 15, said apparatus including emitters which create a corona discharge or similar field, where the emitters include one or more conductive electrodes which are encapsulated or laminated by dielectric material, including glass or ceramic or epoxy filled glass, so that the electrodes are not exposed or adjacent to the gas flow.

17. Apparatus for cleaning and purifying refrigeration systems as defined in any of claims 1 to 16, where the whole apparatus or part of the apparatus is encapsulated inside a potted mass, such as urethane or epoxy or similar material.

18. A method of cleaning and purifying water or surfaces or air in refrigeration systems or an apparatus as defined in any one of claims 1 to 17 characterised in that the refrigeration systems are selected from systems including refrigerated machines or spaces: such as ice machines, chilled water machines, drinking water coolers, cold rooms, cool rooms, refrigerated transport containers, reefers, soft ice machines, ice cream machines, refrigerated trucks, household refrigerators and commercial refrigerators.

19. An apparatus for cleaning and purifying air or water or surfaces in refrigeration systems as described in any one of claims 1 to 18 where the apparatus is a fully potted or encapsulated component which is located inside a refrigeration system, including being located inside the refrigeration unit of a refrigerated container, or inserted into the outside surface of a refrigerated containers refrigeration unit, or inside an ice machine.
